(19) United States
(12) Patent Application Publication
(10) Pub. No.: US 2025/0381125 A1
(43) Pub. Date: Dec. 18, 2025

(54) WASHING COMPOSITION FOR KERATIN FIBRES COMPRISING AT LEAST ONE ANIONIC SURFACTANT, AT LEAST ONE AMPHOTERIC OR ZWITTERIONIC SURFACTANT, AT LEAST ONE GLUCAMIDE COMPOUND AND AT LEAST ONE CATIONIC GALACTOMANNAN GUM

(71) Applicant: L'Oreal, Paris (FR)

(72) Inventors: Fiona Delhaye, Paris (FR); Saber Maloug, Paris (FR)

(73) Assignee: L'Oreal, Paris (FR)

(21) Appl. No.: 18/877,389

(22) PCT Filed: Jun. 22, 2023
(86) PCT No.: PCT/EP2023/066931

§ 371 Date: Dec. 20, 2024

(30) Foreign Application Priority Data
Jun. 22, 2022 (FR) FR2206167

(51) Int. Cl. A61K 8/73; A61Q 5/02; A61Q 5/12

(52) U.S. Cl. CPC A61K 8/737; A61Q 5/02; A61Q 5/12; A61K 2800/34; A61K 2800/43; A61K 2800/5426; A61K 2800/596

(57) ABSTRACT

The present invention relates to a composition comprising at least one anionic surfactant, at least one amphoteric or zwitterionic surfactant, at least one glucamide compound and at least one cationic galactomannan gum, with a weight particular weight ratio of the total content of anionic surfactant(s) (i) to the total content of amphoteric or zwitterionic surfactant(s) (ii). The invention relates to a process for treating keratin fibres, comprising at least one step of applying to said keratin fibres a composition according to the invention.

The present invention relates to a composition comprising at least one anionic surfactant, at least one amphoteric or zwitterionic surfactant, at least one glucamide compound and at least one cationic galactomannan gum, with a particular weight ratio of the total content of anionic surfactant(s) (i) to the total content of amphoteric or zwitterionic surfactant(s) (ii).

The invention relates to a process for treating keratin fibres, comprising at least one step of applying to said keratin fibres a composition according to the invention.

It is common practice to use detergent compositions (such as shampoos) based essentially on surfactants, for cleansing and/or washing keratin fibres such as the hair. These compositions are applied to wet hair and the foam generated by massaging or rubbing with the hands makes it possible, after rinsing with water, to remove the diverse types of soiling initially present on the hair or the skin.

In addition, it is known practice to propose shampoos, in particular for hair that is sensitized or embrittled to various degrees following the action of atmospheric agents or of repeated mechanical or chemical treatments, containing conditioning agents such as silicones, in order to confer conditioning properties on the keratin fibres, for example a better feel, greater manageability or else easy disentangling.

However, these washing compositions based on surfactants and silicones have several drawbacks: the environmental profile of the composition (biodegradability, water footprint) is not always optimal, due to the presence of silicone; the start of foaming, the quality and more particularly the amount of foam generated are judged to be insufficient; rapid regreasing of the keratin fibres, accompanied by lankness.

In addition, repeated applications of these compositions often have the effect of giving the hair an unpleasant feel, loss of volume and liveliness of the head of hair, and occasionally lack of sheen.

These observations gave rise to the interest in developing a natural and biodegradable cosmetic composition which is intended for the treatment of keratin fibres, which is advantageously silicone-free, which has improved working qualities and good cosmetic performance, and which is capable of washing keratin fibres without making them lank, so as to give the keratin fibres good conditioning properties.

In parallel, increasing numbers of users of cosmetic products are seeking hair hygiene products that are more aesthetic or even more original.

There is therefore a real need to develop aesthetic compositions which are stable over time, which are based on natural and/or biodegradable ingredients, which preferably do not comprise silicone, and which make it possible to obtain excellent washing and working properties, in particular in terms of start of foaming, of quality and amount of foam generated, and of viscosity. It is also advantageous for these compositions to confer good conditioning properties on the keratin fibres, in particular in terms of feel, smoothness, manageability and disentangling.

These objectives are achieved by the present invention, one subject of which is a composition, preferably a cosmetic composition, comprising:
(i) at least one anionic surfactant,
(ii) at least one amphoteric or zwitterionic surfactant,
(iii) at least one glucamide compound, and
(iv) at least one cationic galactomannan gum; and
   in which the weight ratio of the total content of anionic surfactant(s) (i) to the total content of amphoteric or zwitterionic surfactant(s) (ii) is less than or equal to 4.3.

It has been found that the composition according to the invention has excellent keratin fibre-washing power.

In particular, the hair treated with the composition according to the invention is particularly clean and has good cosmetic properties.

It has also been noted that the hair thus treated is particularly light, soft to the touch, smooth to the touch, supple, easy to disentangle and more manageable.

In addition, it has been observed that the composition according to the invention has good working qualities, notably a good start of foaming and good foam quality and amount. It has very particularly been noted that the composition according to the invention makes it possible to generate a large amount of foam.

The composition according to the invention has a viscosity suitable for use as a shampoo.

Moreover, it is also be noted that the composition according to the invention is particularly stable over time, at atmospheric pressure and 25° C.

More particularly, when the composition according to the invention also comprises at least one opacifier, the composition remains homogeneous and no phenomenon of phase separation of the composition was observed.

According to one advantageous embodiment of the invention, the composition according to the invention also comprises one or more particles (e.g. opacifiers, pigments, flakes, etc. . . . ), in the form of a homogeneous and stable suspension.

The visually stable and homogeneous suspension of these particles substantially improves the aesthetic appearance and the originality of the composition according to the invention.

For the purposes of the invention, the expression “stable and homogeneous suspension” is intended to mean that said particles are homogeneously dispersed in the composition, that is to say homogeneously distributed within the composition, and that this homogeneous dispersion persists over time, that is to say that the particles do not float at the surface, do not run, do not separate out and/or do not form agglomerates in the composition over time, in particular after at least one week of storage.

A subject of the invention is also a process for treating, in particular cosmetically treating, keratin fibres, in particular human keratin fibres such as the hair, comprising at least one step of applying a composition according to the invention to said keratin fibres.

Other subjects, characteristics, aspects and advantages of the invention will emerge even more clearly on reading the description and the example that follows.

In the present description, and unless otherwise indicated:
the expression “at least one” is equivalent to the expression “one or more” and can be replaced therewith;
the expression “between . . . and . . . ” is equivalent to the expression “ranging from . . . to . . . ” and can be replaced therewith, and implies that the limits are included;
for the purposes of the present invention, the term “less than” and, respectively, the term “greater than” refer to an open range which is strictly less, or, respectively, strictly greater, and thus that the limits are not included;
the term “keratin fibres”, according to the present application, more particularly denotes human keratin fibres, and more preferentially the hair;
the term “fatty acid” according to the present application is intended to mean an organic acid comprising in its structure a linear or branched, saturated or unsaturated hydrocarbon-based chain comprising from 6 to 40 carbon atoms, preferably from 8 to 30 carbon atoms, more preferentially from 10 to 22 carbon atoms;
the term “fatty alcohol” according to the present application is intended to mean an alcohol comprising in its structure a linear or branched, saturated or unsaturated hydrocarbon-based chain comprising from 6 to 40 carbon atoms, preferably from 8 to 30 carbon atoms, more preferentially from 10 to 22 carbon atoms.

Preferably, the composition according to the invention is silicone-free.

The term “silicone-free” is intended to mean that the composition according to the invention does not comprise any silicone, or that the silicone(s) present in the composition according to the invention are included in a total content of less than or equal to 0.1% by weight, preferably less than or equal to 0.05% by weight, more preferentially less than or equal to 0.01% by weight, relative to the total weight of the composition according to the invention, and better still is free of silicone (0% by weight).

The term “silicone” is intended to mean any organosilicon polymer or oligomer of linear or cyclic and branched or crosslinked structure, of variable molecular weight, obtained for example by polymerization and/or polycondensation of suitably functionalized silanes and constituted essentially of a repetition of main units in which the silicon atoms are connected to each other via oxygen atoms (siloxane bond —Si—O—Si—), optionally substituted hydrocarbon-based radicals being connected directly to said silicon atoms via a carbon atom; and more particularly dialkylsiloxane polymers, amino silicones and dimethiconols.

International standard ISO 16128-2 describes the methods for calculating the Natural, Natural Origin, Organic and Organic Origin indexes that apply to the ingredient categories and cosmetic products defined in standard ISO 16128-1. ISO 16128-2 also offers a framework for determining the natural content, natural origin, organic and organic origin content of cosmetic products based on the ingredient characterization.

Preferably, the composition according to the invention has a percentage of natural origin, within the meaning of international standard ISO 16128-2, which is greater than or equal to 90%; more preferentially greater than or equal to 92%; even more preferentially greater than or equal to 94%.

### Anionic Surfactants

The composition according to the present invention comprises at least one anionic surfactant.

The term “anionic surfactant” means a surfactant, preferably a non-silicone surfactant, which includes, as ionic or ionizable groups, only anionic groups.

In the present description, a species is termed as being “anionic” when it bears at least one permanent negative charge or when it can be ionized to a negatively charged species, under the conditions of use of the composition of the invention (for example the medium or the pH) and not comprising any cationic charge.

The anionic surfactants can be chosen from sulfate, sulfonate and carboxylic (or carboxylate) surfactants. Needless to say, a mixture of these surfactants may be used.

It is understood in the present description that:
the carboxylate anionic surfactants comprise at least one carboxylic or carboxylate function (—COOH or —COO⁻) and may optionally also comprise one or more sulfate and/or sulfonate functions;
the sulfonate anionic surfactants comprise at least one sulfonate function (—SO₃H or —SO₃⁻) and may optionally also comprise one or more sulfate functions, but do not comprise any carboxylate functions; and
the sulfate anionic surfactants comprise at least one sulfate function but do not comprise any carboxylate or sulfonate functions.

The carboxylate anionic surfactants that may be used thus include at least one carboxylic or carboxylate function (—COOH or —COO⁻).

The carboxylate anionic surfactants may be chosen from the following compounds: acylglycinates, acyllactylates, acylsarcosinates, acylglutamates; alkyl-D-galactosideuronic acids, alkyl ether carboxylic acids, alkyl(C₆-C₃₀ aryl) ether carboxylic acids, alkylamido ether carboxylic acids; and also the salts of these compounds; and mixtures thereof;
the alkyl and/or acyl groups of these compounds including from 6 to 30 carbon atoms, notably from 12 to 28, even better still from 14 to 24 or even from 16 to 22 carbon atoms; the aryl group preferably denoting a phenyl or benzyl group;
these compounds possibly being polyoxyalkylenated, notably polyoxyethylenated, and then preferably including from 1 to 50 ethylene oxide units and better still from 2 to 10 ethylene oxide units.

Use may also be made of C₆-C₂₄ alkyl monoesters of polyglycoside-polycarboxylic acids such as C₆-C₂₄ alkyl polyglycoside-citrates, C₆-C₂₄ alkyl polyglycoside-tartrates and C₆-C₂₄ alkyl polyglycoside-sulfosuccinates, and salts thereof.

Preferentially, the carboxylate anionic surfactants are chosen, alone or as a mixture, from:
acyl glutamates, notably C₆-C₂₄ or even C₁₂-C₂₀ acyl glutamates, such as stearoyl glutamates, and in particular disodium stearoyl glutamate;
acyl sarcosinates, notably C₆-C₂₄ or even C₁₂-C₂₀ acyl sarcosinates, such as palmitoyl sarcosinates, and in particular sodium palmitoyl sarcosinate;
acyl lactylates, notably C₁₂-C₂₈ or even C₁₄-C₂₄ acyl lactylates, such as behenoyl lactylates, and in particular sodium behenoyl lactylate;
C₆-C₂₄ and notably C₁₂-C₂₀ acylglycinates;
(C₆-C₂₄)alkyl ether carboxylates, and notably (C₁₂-C₂₀)alkyl ether carboxylates;
polyoxyalkylenated (C₆-C₂₄)alkyl(amido) ether carboxylic acids, in particular those including from 2 to 50 ethylene oxide groups;

in particular in the form of alkali metal or alkaline-earth metal, ammonium or amino alcohol salts.

Among the above carboxylic surfactants, mention may be made most particularly of surfactants of sarcosinate type, notably chosen from (C₆-C₃₀)acyl sarcosinates of formula (I) below:

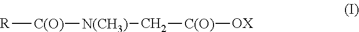

with
X denoting a hydrogen atom, an ammonium ion, an ion derived from an alkali metal or an alkaline-earth metal or an ion derived from an organic amine, preferably a hydrogen atom, and
R denoting a linear or branched alkyl group of 5 to 29 carbon atoms.

Preferably, R denotes a linear or branched alkyl group of 8 to 24 carbon atoms, preferably of 12 to 20 carbon atoms.

Among the (C₆-C₃₀)acyl sarcosinates of formula (I) that may be used in the present composition, mention may be made of palmitoyl sarcosinates, stearoyl sarcosinates, myristoyl sarcosinates, lauroyl sarcosinates and cocoyl sarcosinates, in acid form or in salified form.

The anionic surfactant(s) of sarcosinate type are advantageously chosen from sodium lauroyl sarcosinate, stearoylsarcosine, myristoylsarcosine, and mixtures thereof, preferably from stearoylsarcosine, myristoylsarcosine, and mixtures thereof.

Among the above carboxylic surfactants, mention may also be made of polyoxyalkylenated alkyl (amido) ether carboxylic acids and salts thereof, in particular those including from 2 to 50 alkylene oxide and in particular ethylene oxide groups, such as the compounds sold by Kao under the Akypo names.

The polyoxyalkylenated alkyl (amido) ether carboxylic acids that may be used are preferably chosen from those of formula (II):

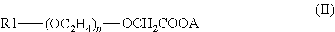

in which:
R1 represents a linear or branched C₆-C₂₄ alkyl or alkenyl radical, a (C₈-C₉)alkylphenyl radical, a radical R₂CONH—CH₂—CH₂— with R2 denoting a linear or branched C₉-C₂₁ alkyl or alkenyl radical;
preferably, R1 is a C₈-C₂₀ and preferably C₈-C₁₈ alkyl radical, and aryl preferably denotes phenyl,
n is an integer or decimal number (mean value) ranging from 2 to 24 and preferably from 2 to 10,
A denotes H, ammonium, Na, K, Li, Mg or a monoethanolamine or triethanolamine residue.

Use may also be made of mixtures of compounds of formula (II), in particular mixtures of compounds bearing different groups R1.

The polyoxyalkylenated alkyl(amido) ether carboxylic acids that are particularly preferred are those of formula (II) in which:
R1 denotes a C₁₂-C₁₄ alkyl, cocoyl, oleyl, nonylphenyl or octylphenyl radical,
A denotes a hydrogen or sodium atom, and
n ranges from 2 to 20, preferably from 2 to 10.

Even more preferentially, use is made of the compounds of formula (II) in which R1 denotes a C₁₂ alkyl radical, A denotes a hydrogen or sodium atom and n ranges from 2 to 10.

The sulfonate anionic surfactants that may be used include at least one sulfonate function (—SO₃H or —SO₃).

The sulfonate anionic surfactants may be chosen from the following compounds: alkylsulfonates, alkylamidesulfonates, alkylarylsulfonates, α-olefin sulfonates, paraffin sulfonates, alkyl sulfosuccinates, alkyl ether sulfosuccinates, alkylamidesulfosuccinates, alkylsulfoacetates, N-acyltaurates, acylisethionates; alkylsulfolaurates; and also the salts of these compounds;
the alkyl groups of these compounds including from 6 to 30 carbon atoms, notably from 12 to 28, even better still from 14 to 24 or even from 16 to 22 carbon atoms; the aryl group preferably denoting a phenyl or benzyl group;
these compounds possibly being polyoxyalkylenated, notably polyoxyethylenated, and then preferably including from 1 to 50 ethylene oxide units and better still from 2 to 10 ethylene oxide units.

Preferentially, the sulfonate anionic surfactants are chosen, alone or as a mixture, from:
C₆-C₂₄ and notably C₁₂-C₂₀ alkyl sulfosuccinates, notably lauryl sulfosuccinates;
C₆-C₂₄ and notably C₁₂-C₂₀ alkyl ether sulfosuccinates;
C₆-C₂₄ and notably C₁₂-C₂₀ N-acyltaurates;
(C₆-C₂₄)acyl isethionates, preferably (C₁₂-C₁₈)acyl isethionates;
in particular in the form of alkali metal or alkaline-earth metal, ammonium or amino alcohol salts.

Preferably, the anionic surfactant(s) of sulfonate type are chosen from C₆-C₂₄ and notably C₁₂-C₂₀ N-acyltaurates, and in particular N-acyl N-methyltaurates, C₆-C₂₄ and notably C₁₂-C₁₈ acylisethionates, and also salts thereof and mixtures thereof.

More preferentially, the anionic surfactant(s) of sulfonate type are chosen from C₆-C₂₄ and notably C₁₂-C₁₈ acylisethionates, and also salts thereof and mixtures thereof.

The sulfate anionic surfactants that may be used include at least one sulfate function (—OSO₃H or —OSO₃⁻).

The sulfate anionic surfactants be chosen from the following compounds: alkyl sulfates, alkyl ether sulfates, alkylamido ether sulfates, alkylaryl polyether sulfates, monoglyceride sulfates; and the salts of these compounds;
the alkyl groups of these compounds including from 6 to 30 carbon atoms, notably from 8 to 28, even better still from 10 to 24 or even from 12 to 22 carbon atoms; the aryl group preferably denoting a phenyl or benzyl group;
these compounds possibly being (poly)oxyalkylenated, notably (poly)oxyethylenated, and then preferably including from 1 to 50 ethylene oxide units and better still from 1 to 10 ethylene oxide units.

Preferentially, the sulfate anionic surfactants are chosen, alone or as a mixture, from:
alkyl sulfates, notably C₁₀-C₂₄ or even C₁₂-C₂₂ alkyl sulfates;
alkyl ether sulfates, notably C₁₀-C₂₄ or even C₁₂-C₂₂ alkyl ether sulfates, preferably comprising from 1 to 20 ethylene oxide units;
in particular in the form of alkali metal or alkaline-earth metal, ammonium or amino alcohol salts.

When the anionic surfactant is in salt form, said salt may be chosen from alkali metal salts, such as the sodium or potassium salt, ammonium salts, amine salts and in particular amino alcohol salts, and alkaline-earth metal salts, such as the magnesium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular sodium or magnesium salts are preferably used.

Advantageously, the anionic surfactant(s) are chosen from sulfate anionic surfactants.

More preferentially, the anionic surfactant(s) are chosen from alkyl sulfates, alkyl ether sulfates, alkylamido ether sulfates, alkylaryl polyether sulfates, monoglyceride sulfates, the salts of these compounds, and mixtures thereof;
the alkyl groups of these compounds preferably comprising from 6 to 30 carbon atoms, notably from 8 to 28, even better still from 10 to 24 or even from 12 to 22 carbon atoms; the aryl group preferably denoting a phenyl or benzyl group;
these compounds possibly being (poly)oxyalkylenated, notably (poly)oxyethylenated, and then preferably including from 1 to 50 ethylene oxide units and better still from 1 to 10 ethylene oxide units.

Even more preferentially, the anionic surfactant(s) are chosen from:
C₆-C₃₀, better still C₈-C₂₄, even better still C₁₀-C₂₄ or even C₁₂-C₂₂ alkyl sulfates,
C₆-C₂₄, better still C₁₀-C₂₄ or even C₁₂-C₂₂ alkyl ether sulfates, preferably comprising from 1 to 20 ethylene oxide units,
the salts of these compounds and mixtures thereof;
in particular in the form of alkali metal or alkaline-earth metal, ammonium or amino alcohol salts.

Even more preferentially, the anionic surfactant(s) are chosen from C₆-C₃₀, better still C₈-C₂₄, even better still C₁₀-C₂₄ or even C₁₂-C₂₂ alkyl sulfates.

Preferably, the total content of anionic surfactant(s) present in the composition ranges from 0.1% to 25% by weight, more preferentially from 1% to 20% by weight, even more preferentially from 2% to 15% by weight and better still from 5% to 10% by weight relative to the total weight of the composition.

Preferably, the total content of sulfate anionic surfactant(s) present in the composition ranges from 0.1% to 25% by weight, more preferentially from 1% to 20% by weight, even more preferentially from 2% to 15% by weight and better still from 5% to 10% by weight relative to the total weight of the composition.

Preferably, the total content of C₆-C₃₀ alkyl sulfates present in the composition ranges from 0.1% to 25% by weight, more preferentially from 1% to 20% by weight, even more preferentially from 2% to 15% by weight, better still from 5% to 10% by weight, relative to the total weight of the composition.

### Amphoteric Surfactants

The composition according to the present invention comprises at least one amphoteric or zwitterionic surfactant.

In particular, the amphoteric or zwitterionic surfactant(s), which are preferably non-silicone, used in the composition according to the present invention may notably be derivatives of optionally quaternized aliphatic secondary or tertiary amines, in which derivatives the aliphatic group is a linear or branched chain including from 8 to 22 carbon atoms, said amine derivatives containing at least one anionic group, for instance a carboxylate, sulfonate, sulfate, phosphate or phosphonate group.

Mention may in particular be made of (C₈-C₂₀)alkylbetaines, (C₈-C₂₀)alkylsulfobetaines, (C₈-C₂₀)alkylamido (C₁-C₆)alkylbetaines and (C₈-C₂₀)alkylamido (C₁-C₆)alkylsulfobetaines, and mixtures thereof.

Among the optionally quaternized derivatives of secondary or tertiary aliphatic amines that may be used, as defined above, mention may also be made of the compounds having the respective structures (III) and (IV) below:

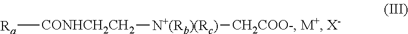

in which formula (III):
Rₐ represents a C₁₀ to C₃₀ alkyl or alkenyl group derived from an acid RₐCOOH preferably present in hydrolyzed copra oil; preferably, Rₐ represents a heptyl, nonyl or undecyl group;
R_{b} represents a β-hydroxyethyl group;
R_{c} represents a carboxymethyl group;
M⁺ represents a cationic counterion derived from an alkali metal or alkaline-earth metal, such as sodium, an ammonium ion or an ion derived from an organic amine; and
X⁻ represents an organic or mineral anionic counterion, such as that chosen from halides, acetates, phosphates, nitrates, (C₁-C₄)alkyl sulfates, (C₁-C₄)alkyl- or (C₁-C₄)alkylaryl- sulfonates, in particular methyl sulfate and ethyl sulfate; or alternatively M⁺ and X⁻ are absent;

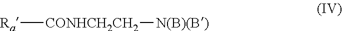

in which formula (IV):
B represents the group —CH₂CH₂OX′;
B′ represents the group —(CH₂)_{z}Y′, with z=1 or 2;
X′ represents the group —CH₂COOH, —CH₂—COOZ′, —CH₂CH₂COOH or CH₂CH₂—COOZ′, or a hydrogen atom;
Y′ represents the group —COOH, —COOZ′ or—CH₂CH(OH)SO₃H or the group CH₂CH(OH)SO₃—Z′;
Z′ represents a cationic counterion derived from an alkali metal or alkaline-earth metal, such as sodium, an ammonium ion or an ion derived from an organic amine;
Rₐ′ represents a C₁₀ to C₃₀ alkyl or alkenyl group of an acid Rₐ′—COOH which is preferably present in coconut kernel oil or in hydrolyzed linseed oil, preferably Rₐ′ an alkyl group, notably a C₁₇ group, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by Rhodia under the trade name Miranol® C2M Concentrate.

Use may also be made of compounds of formula (V):

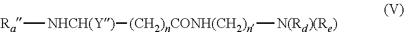

in which formula (V):
Y″ represents the group —COOH, —COOZ″ or —CH₂CH(OH)SO₃H or the group CH₂CH(OH)SO₃—Z″;
R_{d} and Rₑ, independently of each other, represent a C₁ to C₄ alkyl or hydroxyalkyl radical;
Z″ represents a cationic counterion derived from an alkali metal or alkaline-earth metal, such as sodium, an ammonium ion or an ion derived from an organic amine;
Rₐ″ represents a C₁₀ to C₃₀ alkyl or alkenyl group of an acid Rₐ″—COOH which is preferably present in coconut kernel oil or in hydrolyzed linseed oil; and
n and n′ denote, independently of each other, an integer ranging from 1 to 3.

Among the compounds of formula (V), mention may be made of the compound classified in the CTFA dictionary under the name sodium diethylaminopropyl cocoaspartamide and sold by Chimex under the name Chimexane HB.

These compounds may be used alone or as mixtures.

Among the amphoteric or zwitterionic surfactants mentioned above, use is advantageously made of (C₈-C₂₀)alkylbetaines, such as cocobetaine, (C₈-C₂₀)alkylamido (C₃-C₈)alkylbetaines, such as cocamidopropylbetaine, (C₈-C₂₀)alkylamphoacetates, (C₈-C₂₀)alkylamphodiacetates and mixtures thereof.

More preferentially, the amphoteric or zwitterionic surfactant(s) are chosen from (C₈-C₂₀)alkylbetaines, (C₈-C₂₀)alkylamido (C₃-C₈)alkylbetaines, and mixtures thereof; even more preferentially from cocobetaine, cocamidopropylbetaine, and mixtures thereof.

Better still, the amphoteric or zwitterionic surfactants are chosen from (C₈-C₂₀)alkylamido (C₃-C₈)alkylbetaines and most particularly cocamidopropylbetaine.

Preferably, the total content of amphoteric or zwitterionic surfactant(s) present in the composition ranges from 0.1% to 15% by weight, more preferentially from 0.5% to 10% by weight, even more preferentially from 1% to 5% by weight relative to the total weight of the composition.

Preferably, the total content of (C₈-C₂₀)alkylbetaine(s) and (C₈-C₂₀)alkylamido(C₃-C₈)alkylbetaine(s) present in the composition ranges from 0.1% to 15% by weight, more preferentially from 0.5% to 10% by weight, even more preferentially from 1% to 5% by weight relative to the total weight of the composition.

Preferably, the total content of (C₈-C₂₀)alkylamido (C₃-C₈)alkylbetaine(s), such as cocamidopropylbetaine, present in the composition ranges from 0.1% to 15% by weight, more preferentially from 0.5% to 10% by weight, even more preferentially from 1% to 5% by weight relative to the total weight of the composition.

Advantageously, the total content of anionic surfactant(s) and amphoteric or zwitterionic surfactant(s) present in the composition is greater than or equal to 2% by weight, and more preferentially greater than or equal to 4% by weight, relative to the total weight of the composition.

Preferably, the total content of anionic surfactant(s) and amphoteric or zwitterionic surfactant(s) present in the composition ranges from 4% to 40% by weight, more preferentially from 5% to 25% by weight, even more preferentially from 6% to 20% by weight, better still from 7% to 15% by weight and even better still from 7.5% to 12% by weight, relative to the total weight of the composition.

Preferably, the total content of sulfate anionic surfactant(s) and amphoteric or zwitterionic surfactant(s) present in the composition ranges from 4% to 40% by weight, more preferentially from 5% to 25% by weight, even more preferentially from 6% to 20% by weight, better still from 7% to 15% by weight and even better still from 7.5% to 12% by weight, relative to the total weight of the composition.

Preferably, the total content of C₆-C₃₀ alkyl sulfates and (C₈-C₂₀)alkylbetaine(s) and (C₈-C₂₀)alkylamido(C₃-C₈)alkylbetaine(s) present in the composition ranges from 4% to 40% by weight, more preferentially from 5% to 25% by weight, even more preferentially from 6% to 20% by weight, better still from 7% to 15% by weight and even better still from 7.5% to 12% by weight, relative to the total weight of the composition.

According to the invention, the weight ratio of the total content of anionic surfactant(s) (i) to the total content of amphoteric or zwitterionic surfactant(s) (ii) is less than or equal to 4.3.

Preferably, the weight ratio of the total content of anionic surfactant(s) (i) to the total content of amphoteric and zwitterionic surfactant(s) (ii) is between 1 and 4.3; more preferentially between 1.5 and 4.3; even more preferentially less than or equal to 4; better still between 2 and 4; and even better still between 2.1 and 3.9.

Preferably, the weight ratio of the total content of sulfate anionic surfactant(s) to the total content of amphoteric and zwitterionic surfactant(s) is between 1 and 4.3; more preferentially between 1.5 and 4.3; even more preferentially less than or equal to 4; better still between 2 and 4; and even better still between 2.1 and 3.9.

Preferably, the weight ratio of the total content of C₆-C₃₀, alkyl sulfates to the total content of (C₈-C₂₀)alkylbetaine(s) and (C₈-C₂₀)alkylamido (C₃-C₈)alkylbetaine(s) is between 1 and 4.3; more preferentially between 1.5 and 4.3; even more preferentially less than or equal to 4; better still between 2 and 4; and even better still between 2.1 and 3.9.

### Glucamide Compounds

The composition according to the invention comprises at least one glucamide compound.

The glucamide compounds which can be used according to the invention are notably described in patent applications WO 92 06154, U.S. Pat. No. 5,194,639 and DE 44 43 645.

Preferably, the glucamide compounds are chosen from acylglucamides, more preferentially those having a hydrocarbon-based chain comprising from 4 to 30 carbon atoms (counting the carbon atom of the —C(O)— carbonyl group), preferentially from 6 to 22, better still from 6 to 20 and even better still from 6 to 14.

More preferentially, the glucamide compounds are chosen from those of general formula (A) below:

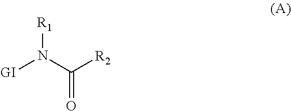

in which:
the Gl—N group represents a glucamine, of which N is the nitrogen atom of the glucamine,
R₁ represents a C₁-C₂ alkyl radical, preferably a methyl,
R₂ represents a linear or branched, C₃-C₂₉, preferably C₅-C₂₁, more preferentially C₅-C₁₉, better still C₅-C₁₃, alkyl or alkenyl radical which is optionally substituted with one or more hydroxyl —OH groups.

Even more preferentially, in formula (A) above:
the Gl—N group represents a glucamine, of which N is the nitrogen atom of the glucamine,
R₁ represents a methyl, and
R₂ represents a linear C₃-C₂₉, preferably C₅-C₂₁, more preferentially C₅-C₁₉, better still C₅-C₁₃, even better still C₇-C₉ alkyl radical which is optionally substituted with one or more hydroxyl —OH groups.

Advantageously, the composition according to the invention can comprise at least two glucamide groups; more preferentially, the composition according to the invention comprises two glucamide groups.

For the purposes of the present invention, the terms “glucamide” and “glucamine” encompass all of the respective isomers thereof.

Among the glucamide compounds that may be used according to the invention, mention may in particular be made, alone or as a mixture, of:
myristoyl methylglucamide, lauroyl methylglucamide, capryloyl methylglucamide, caproyl methylglucamide, cocoyl methylglucamide, nonanoyl methylglucamide, oleyl methylglucamide, sunfloweroyl methylglucamide,
oleoyl methylglucamide oleate, stearoyl methylglucamide stearate, tocopheryl methylglucamide succinate.

Preference is given very particularly to the glucamide compounds chosen from, alone or as a mixture, myristoyl methylglucamide, lauroyl methylglucamide, capryloyl methylglucamide, caproyl methylglucamide, cocoyl methylglucamide, nonanoyl methylglucamide, oleyl methylglucamide and sunfloweroyl methylglucamide.

Among the mixtures of glucamide compounds, mention may be made of lauroyl/myristoyl methylglucamide and capryloyl/caproyl methylglucamide which are particularly preferred.

Even more preferentially, the composition according to the invention comprises at least capryloyl/caproyl methylglucamide.

Preferably, the total content of glucamide compound(s) present in the composition ranges from 0.01% to 10% by weight, more preferentially from 0.5% to 5% by weight, even more preferentially from 0.8% to 3% by weight relative to the total weight of the composition.

Preferably, the total content of acylglucamide compound(s) present in the composition ranges from 0.01% to 10% by weight, more preferentially from 0.5% to 5% by weight, even more preferentially from 0.8% to 3% by weight relative to the total weight of the composition.

Preferably, the total content of acylglucamide compound(s) of which the hydrocarbon-based chain comprises between 4 and 30 carbon atoms, present in the composition, ranges from 0.01% to 10% by weight, more preferentially from 0.5% to 5% by weight, even more preferentially from 0.8% to 3% by weight relative to the total weight of the composition.

Preferably, the total content of glucamide compound(s) of formula (A) present in the composition ranges from 0.01% to 10% by weight, more preferentially from 0.5% to 5% by weight, even more preferentially from 0.8% to 3% by weight relative to the total weight of the composition.

Preferably, the total content of capryloyl/caproyl methylglucamide in the composition ranges from 0.01% to 10% by weight, more preferentially from 0.5% to 5% by weight, even more preferentially from 0.8% to 3% by weight relative to the total weight of the composition.

### Non-Ionic Surfactants

Preferably, the composition according to the present invention also comprises at least one non-ionic surfactant.

More preferentially, the composition according to the present invention also comprises at least two non-ionic surfactants.

By way of examples, the non-ionic surfactants that can be used according to the invention may be chosen from:
alcohols, α-diols and (C₁-C₂₀)alkylphenols, these compounds being polyethoxylated and/or polypropoxylated and/or polyglycerolated, the number of ethylene oxide and/or propylene oxide groups possibly ranging from 1 to 100, and the number of glycerol groups possibly ranging from 2 to 30; or else these compounds comprising at least one fatty chain including from 8 to 40 carbon atoms and notably from 16 to 30 carbon atoms; in particular, oxyethylenated alcohols comprising at least one saturated or unsaturated, linear or branched C₈ to C₄₀ alkyl chain, comprising from 1 to 100 mol of ethylene oxide, preferably from 2 to 50 and more particularly from 2 to 40 mol of ethylene oxide and including one or two fatty chains;
condensates of ethylene oxide and propylene oxide with fatty alcohols;
polyethoxylated fatty amides preferably containing from 2 to 30 ethylene oxide units, polyglycerolated fatty amides including on average from 1 to 5 and in particular from 1.5 to 4 glycerol groups;
ethoxylated fatty acid esters of sorbitan, preferably containing from 2 to 40 ethylene oxide units;
fatty acid esters of sucrose;
polyoxyalkylenated, preferably polyoxyethylenated, fatty acid esters containing from 2 to 150 mol of ethylene oxide, including oxyethylenated plant oils;
N—(C₆-C₂₄ alkyl)glucamine derivatives;
amine oxides such as (C₁₀-C₁₄ alkyl)amine oxides or N—(C₁₀-C₁₄ acyl)aminopropylmorpholine oxides;
and mixtures thereof.

Preferably, the non-ionic surfactant(s) are chosen from monoesters of a fatty acid and of (poly)glycerol, alkyl(poly)glycosides, and mixtures thereof.

More preferentially, the non-ionic surfactant(s) are chosen from monoesters of a C₆-C₄₀ fatty acid and of (poly)glycerol, (C₆-C₃₀)alkyl(poly)glycosides, and mixtures thereof.

Among the monoesters of a fatty acid and of (poly)glycerol that can be used according to the invention, mention may in particular be made of monoesters of a fatty acid and of (poly)glycerol comprising on average from 1 to 30 mol of glycerol; more preferentially from 1 to 10 mol of glycerol, better still from 1 to 5 mol of glycerol.

For the purposes of the present invention, the term “average” is intended to mean a number-average.

More preferentially, the monoester(s) of a fatty acid and of (poly)glycerol can be chosen from monoesters of a C₆-C₄₀ fatty acid and of (poly)glycerol; even more preferentially from monoesters of a C₈-C₃₀ fatty acid and of (poly)glycerol.

According to one preferred embodiment of the invention, the monoester(s) of a fatty acid and of (poly)glycerol are chosen from the compounds of formula (X) below:

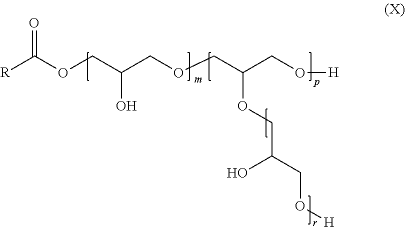

in which:
R represents a linear or branched, saturated or unsaturated C₅-C₃₉, preferably C₇-C₂₉, better still C₁₁-C₂₁, hydrocarbon-based chain;
m, p and r represent an integer between 0 and 30, and are such that the sum of the integers m, p and r is between 1 and 30, better still between 1 and 10, and even better still between 1 and 5.

Preferably, according to this embodiment, p=r=0, and m is an integer between 1 and 30, more preferentially m is an integer between 1 and 10, or even between 1 and 5.

Even more preferentially, the composition according to the present invention also comprises at least one monoester of a fatty acid and of (poly)glycerol; better still at least one monoester of a fatty acid and of glycerol (that is to say among the compounds of formula (X) for which p=r=0 and m=1); even better still at least one monoester of a C₆-C₄₀ (or even C₈-C₃₀) fatty acid and of glycerol.

According to one particularly preferred embodiment of the invention, the monoester(s) of a fatty acid and of (poly)glycerol are chosen from the compounds of formula (XI) below:

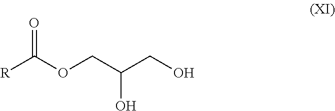

in which:
R represents a linear or branched, saturated or unsaturated C₅-C₃₉, preferably C₇-C₂₉, better still C₁₁-C₂₁ and even better still C₁₄-C₁₉ hydrocarbon-based chain;

Preferably, according to this embodiment, the hydrocarbon-based chain R is unsaturated.

Very particularly preferably, the monoester(s) of a fatty acid and of (poly)glycerol are chosen from the monoesters of a fatty acid and of glycerol (i.e. 1 mol of glycerol); preferably from the monoesters of a C₆-C₄₀ (even better still C₈-C₃₀) and of glycerol; and very particularly from the monoester of oleic acid and of glycerol, the monoester of sapienic acid and of glycerol, the monoester of pamitoleic acid and of glycerol, the monoester of linoleic acid and of glycerol, the monoester of lauric acid and of glycerol, the monoester of myristic acid and of glycerol, the monoester of stearic acid and of glycerol, the monoester of palmitic acid and of glycerol, the monoester of arachidonic acid and of glycerol, and mixtures thereof.

Better still, the monoester(s) of a fatty acid and of (poly)glycerol are chosen from the monoesters of oleic acid and of (poly)glycerol (such as glyceryl oleate, polyglyceryl-2 oleate or polyglyceryl-5 oleate), and mixtures thereof.

Preferably, the total content of monoester(s) of a fatty acid, preferentially a C₆-C₄₀ (even better still C₈-C₃₀) fatty acid, and of (poly)glycerol, when they are present in the composition according to the invention, is between 0.01% and 10% by weight, more preferentially between 0.05% and 8% by weight, even more preferentially between 0.1% and 5%, even better still between 0.1% and 2% by weight, relative to the total weight of the composition.

Preferably, the total content of monoester(s) of a fatty acid, preferentially a C₆-C₄₀ (even better still C₈-C₃₀) fatty acid, and of glycerol, when they are present in the composition according to the invention, is between 0.01% and 10% by weight, more preferentially between 0.05% and 8% by weight, even more preferentially between 0.1% and 5%, even better still between 0.1% and 2% by weight, relative to the total weight of the composition.

Preferably, the total content of glyceryl oleate, when it is present in the composition according to the invention, is between 0.01% and 10% by weight, more preferentially between 0.05% and 8% by weight, even more preferentially between 0.1% and 5% by weight, even better still between 0.1% and 2% by weight, relative to the total weight of the composition.

Among the alkyl (poly)glycosides that can be used according to the invention, mention may be made in particular of the alkyl (poly)glycosides of general formula below:

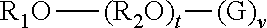

in which:
R₁ represents a linear or branched alkyl or alkenyl radical including 6 to 30 carbon atoms and notably 8 to 24 carbon atoms, or an alkylphenyl radical of which the linear or branched alkyl radical includes 6 to 30 carbon atoms and notably 8 to 24 carbon atoms;
R₂ represents an alkylene radical including 2 to 4 carbon atoms;
G represents a sugar unit including 5 to 6 carbon atoms;
t denotes a value ranging from 0 to 10 and preferably from 0 to 4;
v denotes a value ranging from 1 to 15 and preferably from 1 to 4.

Preferably, the alkyl (poly)glycosides are compounds of the formula described above in which:
R₁ denotes a linear or branched, saturated or unsaturated alkyl radical including from 8 to 24 carbon atoms,
R₂ represents an alkylene radical including 2 to 4 carbon atoms,
t denotes a value ranging from 0 to 3 and preferably equal to 0,
G denotes glucose, fructose or galactose, preferably glucose;
it being possible for the degree of polymerization, i.e. the value of v, to range from 1 to 15 and preferably from 1 to 4; the mean degree of polymerization more particularly being between 1 and 2.

The glucoside bonds between the sugar units are generally of 1-6 or 1-4 type and preferably of 1-4 type.

Advantageously, the alkyl(poly)glycosides are alkyl(poly)glucosides, such as (C₆-C₃₀)alkyl(poly)glucosides. 1,4 (C₈-C₂₄)alkyl(poly)glucosides, and notably coco glucosides, decyl glucosides and caprylyl/capryl glucosides, are most particularly preferred.

Among the commercial products, mention may be made of the products sold by Cognis under the names Plantaren® (600 CS/U, 1200 and 2000) or Plantacare® (818, 1200 and 2000); the products sold by SEPPIC under the names Oramix CG 110 and Oramix® NS 10; the products sold by BASF under the name Lutensol GD 70, or the products sold by Chem Y under the name AG10 LK.

Even more preferentially, the composition according to the present invention also comprises at least one alkyl(poly)glycoside; better still at least one alkyl(poly)glycoside chosen from (C₆-C₃₀)alkyl(poly)glycosides; even better still from (C₈-C₂₄)alkyl(poly)glycosides; or even from (C₈-C₁₈)alkyl(poly)glycosides such as coco glucosides, decyl glucosides, caprylyl/capryl glucosides, lauryl glucosides, and mixtures thereof.

According to one preferred embodiment of the invention, the composition comprises at least one alkyl(poly)glucoside; preferably chosen from (C₆-C₃₀)alkyl(poly)glucosides; even preferentially more from (C₈-C₂₄)alkyl(poly)glucosides; even better still from (C₈-C₁₈)alkyl(poly)glucosides such as coco glucosides, decyl glucosides, caprylyl/capryl glucosides, lauryl glucosides, and mixtures thereof.

Preferably, the total content of alkyl(poly)glycoside(s), when they are present in the composition according to the invention, ranges from 0.01% to 10% by weight, more preferentially from 0.05% to 5% by weight, even more preferentially from 0.1% to 3% by weight and better still from 0.1% to 1% by weight relative to the total weight of the composition.

Preferably, the total content of the (C₆-C₃₀)alkyl(poly)glycoside(s), when they are present in the composition according to the invention, ranges from 0.01% to 10% by weight, more preferentially from 0.05% to 5% by weight, even more preferentially from 0.1% to 3% by weight, even better still from 0.1% to 1% by weight, relative to the total weight of the composition.

Preferably, the total content of the (C₈-C₂₄)alkyl(poly)glycoside(s), when they are present in the composition according to the invention, ranges from 0.01% to 10% by weight, more preferentially from 0.05% to 5% by weight, even more preferentially from 0.1% to 3% by weight, even better still from 0.1% to 1% by weight, relative to the total weight of the composition.

Preferably, the total content of (C₆-C₃₀)alkyl(poly)glucoside(s), when they are present in the composition according to the invention, ranges from 0.01% to 10% by weight, more preferentially from 0.05% to 5% by weight, even more preferentially from 0.1% to 3% by weight and better still from 0.1% to 1% by weight relative to the total weight of the composition.

Preferably, the total content of the (C₈-C₂₄)alkyl(poly)glycoside(s), when they are present in the composition according to the invention, ranges from 0.01% to 10% by weight, more preferentially from 0.05% to 5% by weight, even more preferentially from 0.1% to 3% by weight, even better still from 0.1% to 1% by weight, relative to the total weight of the composition.

Very particularly preferably, the composition according to the invention also comprises:
(iv) at least one monoester of a fatty acid and of (poly)glycerol; preferably chosen from monoesters of a C₆-C₄₀ fatty acid and of (poly)glycerol; more preferentially from monoesters of a C₈-C₃₀ fatty acid and of (poly)glycerol; even more preferentially from monoesters of a C₈-C₃₀ fatty acid and of glycerol, as described above; and
(v) at least one alkyl(poly)glycoside; preferably chosen from (C₆-C₃₀)alkyl(poly)glycosides; more preferentially from (C₈-C₂₄)alkyl(poly)glycosides; even more preferentially from (C₈-C₁₈)alkyl(poly)glycosides; better still from coco glucosides, decyl glucosides, caprylyl/capryl glucosides, lauryl glucosides, and mixtures thereof, as described above.

Preferably, the weight ratio of the total content of alkyl(poly)glycoside(s) (v) to the total content of monoester(s) of a fatty acid and of (poly)glycerol (iv), when they are present in the composition according to the invention, is less than or equal to 1, more preferentially less than 1, even more preferentially less than or equal to 0.9, better still between 0.01 and 0.9, even better still less than or equal to 0.8, or even between 0.05 and 0.8, particularly preferably less than or equal to 0.7, and very particularly preferably between 0.1 and 0.7.

Preferably, the weight ratio of the total content of (C₆-C₃₀)alkyl(poly)glycoside(s) (v) to the total content of monoester(s) of a C₆-C₄₀ (better still C₈-C₃₀) fatty acid and of (poly)glycerol (iv), when they are present in the composition according to the invention, is less than or equal to 1, more preferentially less than 1, even more preferentially less than or equal to 0.9, better still between 0.01 and 0.9, even better still less than or equal to 0.8, or even between 0.05 and 0.8, particularly preferably less than or equal to 0.7, and very particularly preferably between 0.1 and 0.7.

Preferably, the weight ratio of the total content of (C₈-C₂₄)alkyl(poly)glycoside(s) (v) to the total content of monoester(s) of a C₆-C₄₀ fatty acid and of glycerol (iv), when they are present in the composition according to the invention, is less than or equal to 1, more preferentially less than 1, even more preferentially less than or equal to 0.9, better still between 0.01 and 0.9, even better still less than or equal to 0.8, or even between 0.05 and 0.8, particularly preferably less than or equal to 0.7, and very particularly preferably between 0.1 and 0.7.

Preferably, the weight ratio of the total content of (C₆-C₃₀)alkyl(poly)glycoside(s) (v) to the total content of monoester(s) of a C₆-C₄₀ (better still C₈-C₃₀) fatty acid and of glycerol (iv), when they are present in the composition according to the invention, is less than or equal to 1, more preferentially less than 1, even more preferentially less than or equal to 0.9, better still between 0.01 and 0.9, even better still less than or equal to 0.8, or even between 0.05 and 0.8, particularly preferably less than or equal to 0.7, and very particularly preferably between 0.1 and 0.7.

### Cationic Polymers

The composition according to the present invention comprises at least one cationic polymer chosen from cationic galactomannan gums.

According to one embodiment of the invention, the composition may further comprise at least one additional cationic polymer, other than cationic galactomannan gum.

For the purposes of the present invention, the expression “cationic polymer” denotes any non-silicone (not comprising any silicon atoms) polymer containing cationic groups and/or groups that can be ionized into cationic groups and not containing any anionic groups and/or groups that can be ionized into anionic groups.

The cationic polymers are not silicone-based (they do not comprise any Si—O units).

The cationic polymers may be associative or non-associative.

Preferably, the cationic polymer(s) are chosen from non-associative cationic polymers.

The cationic polymers that may be used preferably have a weight-average molar mass (Mw) of between 500 and 5×10⁶ approximately and preferably between 10³ and 3×10⁶ approximately.

Among the cationic polymers, mention may be made more particularly of:
(1) homopolymers or copolymers derived from acrylic or methacrylic esters or amides and including at least one of the units having the following formulae:

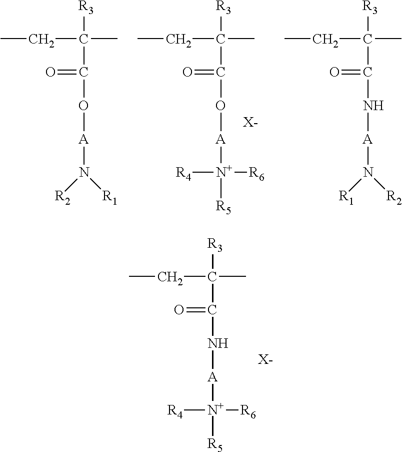

in which formulae:
R₃, which may be identical or different, denote a hydrogen atom or a CH₃ radical;
A, which may be identical or different, represent a linear or branched divalent alkyl group of 1 to 6 carbon atoms, preferably 2 or 3 carbon atoms, or a hydroxyalkyl group of 1 to 4 carbon atoms;
R₄, R₅ and R₆, which may be identical or different, represent an alkyl group containing from 1 to 18 carbon atoms or a benzyl radical, and preferably an alkyl group containing from 1 to 6 carbon atoms;
R₁ and R₂, which may be identical or different, represent a hydrogen atom or an alkyl group containing from 1 to 6 carbon atoms, preferably methyl or ethyl; and
X denotes an anion derived from a mineral or organic acid, such as a methosulfate anion or a halide such as chloride or bromide.

The copolymers of family (1) may also contain one or more units derived from comonomers which may be chosen from the family of acrylamides, methacrylamides, diacetone acrylamides, acrylamides and methacrylamides substituted on the nitrogen with lower (C₁-C₄)alkyls, acrylic acids or methacrylic acids or esters thereof, vinyllactams such as vinylpyrrolidone or vinylcaprolactam, and vinyl esters.

Among these copolymers of family (1), mention may be made of:
copolymers of acrylamide and of dimethylaminoethyl methacrylate quaternized with dimethyl sulfate or with a dimethyl halide, such as the product sold under the name Hercofloc by Hercules,
copolymers of acrylamide and of methacryloyloxyethyltrimethylammonium chloride, such as the products sold under the name Bina Quat P 100 by Ciba Geigy,
the copolymer of acrylamide and of methacryloyloxyethyltrimethylammonium methosulfate, such as the product sold under the name Reten by Hercules,
quaternized or non-quaternized vinylpyrrolidone/dialkylaminoalkyl acrylate or methacrylate copolymers, such as the products sold under the name Gafquat by ISP, for instance Gafquat 734 or Gafquat 755, or alternatively the products known as Copolymer 845, 958 and 937. These polymers are described in detail in French patents 2 077 143 and 2 393 573,
dimethylaminoethyl methacrylate/vinylcaprolactam/vinylpyrrolidone terpolymers, such as the product sold under the name Gaffix VC 713 by ISP,
vinylpyrrolidone/methacrylamidopropyldimethylamine copolymers, such as the products sold under the name Styleze CC 10 by ISP;
quaternized vinylpyrrolidone/dimethylaminopropylmethacrylamide copolymers such as the product sold under the name Gafquat HS 100 by ISP;
polymers, preferably crosslinked polymers, of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homo-or copolymerization being followed by crosslinking with an olefinically unsaturated compound, in particular methylenebisacrylamide. A crosslinked acrylamide/methacryloyloxyethyltrimethylammonium chloride copolymer (20/80 by weight) in the form of a dispersion comprising 50% by weight of said copolymer in mineral oil may more particularly be used. This dispersion is sold under the name Salcare® SC 92 by Ciba. Use may also be made of a crosslinked methacryloyloxyethyltrimethylammonium chloride homopolymer comprising approximately 50% by weight of the homopolymer in mineral oil or in a liquid ester.

These dispersions are sold under the names Salcare® SC 95 and Salcare® SC 96 by Ciba;
(2) cationic polysaccharides, notably cationic celluloses and galactomannan gums. Among the cationic polysaccharides, mention may be made more particularly of cellulose ether derivatives including quaternary ammonium groups, cationic cellulose copolymers or cellulose derivatives grafted with a water-soluble quaternary ammonium monomer and cationic galactomannan gums.

The cellulose ether derivatives including quaternary ammonium groups are notably described in FR 1 492 597, and mention may be made of the polymers sold under the name Ucare Polymer JR (JR 400 LT, JR 125 and JR 30M) or LR (LR 400 and LR 30M) by Amerchol. These polymers are also defined in the CTFA dictionary as quaternary ammoniums of hydroxyethylcellulose that have reacted with an epoxide substituted with a trimethylammonium group, for instance Polyquaternium-10.

Cationic cellulose copolymers or cellulose derivatives grafted with a water-soluble quaternary ammonium monomer are notably described in patent U.S. Pat. No. 4 131 576, and mention may be made of hydroxyalkyl celluloses, for instance hydroxymethyl, hydroxyethyl or hydroxypropyl celluloses notably grafted with a methacryloylethyltrimethylammonium, methacrylamidopropyltrimethylammonium or dimethyldiallylammonium salt, for instance Polyquaternium-4. The commercial products corresponding to this definition are more particularly the products sold under the names Celquat L 200 and Celquat H 100 by National Starch.

Among the cationic cellulose derivatives, use may also be made of cationic associative celluloses, which may be chosen from quaternized cellulose derivatives, and in particular quaternized celluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24, or even from 10 to 14, carbon atoms; or mixtures thereof.

Preferably, mention may be made of quaternized hydroxyethylcelluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24 or even from 10 to 14 carbon atoms; or mixtures thereof.

Preferentially, mention may be made of the hydroxyethylcelluloses of formula (Ib):

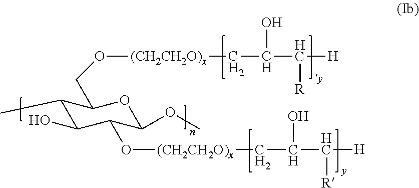

in which:
R represents an ammonium group RaRbRcN⁺—, Q⁻ in which Ra, Rb and Rc, which may be identical or different, represent a hydrogen atom or a linear or branched C₁ to C₃₀ alkyl, preferably an alkyl, and Q⁻ represents an anionic counterion such as a halide, for instance a chloride or bromide;
R′ represents an ammonium group R′aR′bR′cN⁺—, Q′⁻ in which R′a, R′b and R′c, which may be identical or different, represent a hydrogen atom or a linear or branched C₁ to C₃₀ alkyl, preferably an alkyl, and Q′⁻ represents an anionic counterion such as a halide, for instance a chloride or bromide;
it being understood that at least one of the radicals Ra, Rb, Rc, R′a, R′b and R′c represents a linear or branched C₈ to C₃₀ alkyl;
n, x and y, which may be identical or different, represent an integer of between 1 and 10 000.

Preferably, in formula (Ib), at least one of the radicals Ra, Rb, Rc, R′a, R′b or R′c represents a linear or branched C₈ to C₃₀, better still C₁₀ to C₂₄ or even C₁₀ to C₁₄ alkyl; mention may be made in particular of the dodecyl radical (C₁₂). Preferably, the other radical(s) represent a linear or branched C₁-C₄ alkyl, notably methyl.

Preferably, in formula (Ib), only one of the radicals Ra, Rb, Rc, R′a, R′b or R′c represents a linear or branched C₈ to C₃₀, better still C₁₀ to C₂₄ or even C₁₀ to C₁₄ alkyl; mention may be made in particular of the dodecyl radical (C₁₂). Preferably, the other radicals represent a linear or branched C₁ to C₄ alkyl, notably methyl.

Even better still, R may be a group chosen from —N⁺(CH₃)₃, Q′⁻ and
—N⁺(C₁₂H₂₅)(CH₃)₂, Q′⁻ preferably a group —N⁺(CH₃)₃, Q′⁻.

Even better still, R′ may be a group —N⁺(C₁₂H₂₅)(CH₃)₂, Q′⁻.

The aryl radicals preferably denote phenyl, benzyl, naphthyl or anthryl groups.

Mention may notably be made of the polymers having the following INCI names:
Polyquaternium-24, such as the product Quatrisoft LM 200®, sold by Amerchol/Dow Chemical;
PG-Hydroxyethylcellulose Cocodimonium Chloride, such as the product Crodacel QM®;
PG-Hydroxyethylcellulose Lauryldimonium Chloride (C₁₂ alkyl), such as the product Crodacel QL®; and
PG-Hydroxyethylcellulose Stearyldimonium Chloride (C₁₈ alkyl), such as the product Crodacel QS®, sold by Croda.

Mention may also be made of the hydroxyethylcelluloses of formula (Ib) in which R represents a trimethylammonium halide and R′ represents a dimethyldodecylammonium halide, preferentially R represents trimethylammonium chloride (CH₃)₃N⁺—, Cl⁻ and R′ represents dimethyldodecylammonium chloride (CH₃)₂(C₁₂H₂₅)N⁺—, Cl⁻. This type of polymer is known under the INCI name Polyquaternium-67; as commercial products, mention may be made of the Softcat Polymer SL® polymers, such as SL-100, SL-60, SL-30 and SL-5, from Amerchol/Dow Chemical.

More particularly, the polymers of formula (Ib) are, for example, those the viscosity of which is between 2000 and 3000 cPs inclusive, preferentially between 2700 and 2800 cPs. Typically, Softcat Polymer SL-5 has a viscosity of 2500 cPs, Softcat Polymer SL-30 has a viscosity of 2700 cPs, Softcat Polymer SL-60 has a viscosity of 2700 cPs and Softcat Polymer SL-100 has a viscosity of 2800 cPs. Use may also be made of Softcat Polymer SX-1300X with a viscosity of between 1000 and 2000 cPs.

The cationic galactomannan gums are described more particularly in patents U.S. Pat. Nos. 3,589,578 and 4,031,307, and mention may be made of guar gums comprising cationic trialkylammonium groups. Use is made, for example, of guar gums modified with a 2,3-epoxypropyltrimethylammonium salt (for example, a chloride). Such products are notably sold under the names Jaguar C13 S, Jaguar C 15, Jaguar C 17, Jaguar C162 or Jaguar Excel by Rhodia. Such compounds have the INCI name guar hydroxypropyltrimonium chloride or hydroxypropyl guar hydroxypropyltrimonium chloride.
(3) polymers formed from piperazinyl units and divalent alkylene or hydroxyalkylene radicals containing linear or branched chains, optionally interrupted with oxygen, sulfur or nitrogen atoms or with aromatic or heterocyclic rings, and also the oxidation and/or quaternization products of these polymers;
(4) water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyaminoamides can be alkylated or, if they include one or more tertiary amine functions, they can be quaternized;
(5) polyaminoamide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical includes from 1 to 4 carbon atoms and preferably denotes methyl, ethyl or propyl. Among these derivatives, mention may be made more particularly of the adipic acid/dimethylaminohydroxypropyl/diethylenetriamine polymers sold under the name Cartaretine F, F4 or F8 by Sandoz;
(6) polymers obtained by reacting a polyalkylene polyamine including two primary amine groups and at least one secondary amine group with a dicarboxylic acid chosen from diglycolic acid and saturated aliphatic dicarboxylic acids containing from 3 to 8 carbon atoms; the mole ratio between the polyalkylene polyamine and the dicarboxylic acid preferably being between 0.8:1 and 1.4:1; the resulting polyaminoamide being reacted with epichlorohydrin in a mole ratio of epichlorohydrin relative to the secondary amine group of the polyaminoamide preferably of between 0.5:1 and 1.8:1. Polymers of this type are sold in particular under the name Hercosett 57 by Hercules Inc. or under the name PD 170 or Delsette 101 by Hercules in the case of the adipic acid/epoxypropyl/diethylenetriamine copolymer;
(7) cyclopolymers of alkyldiallylamine or of dialkyldiallylammonium, such as homopolymers or copolymers including, as main constituent of the chain, units corresponding to formula (VI) or (VII):

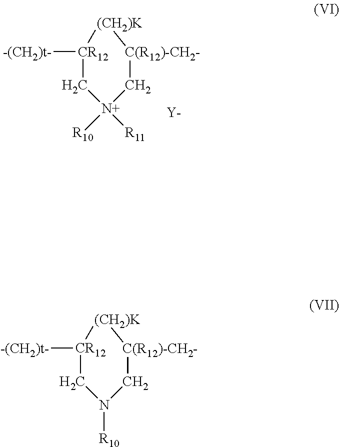

in which formulae (VI) and (VII):
k and t are equal to 0 or 1, the sum k+t being equal to 1;
R₁₂ denotes a hydrogen atom or a methyl radical;
R₁₀ and R₁₁, independently of one another, denote an alkyl group containing from 1 to 6 carbon atoms, a hydroxyalkyl group in which the alkyl group contains 1 to 5 carbon atoms, a C₁ to C₄ amidoalkyl group; or alternatively R₁₀ and R₁₁ may denote, together with the nitrogen atom to which they are attached, heterocyclic groups such as piperidinyl or morpholinyl; R₁₀ and R₁₁, independently of one another, preferably denote an alkyl group containing from 1 to 4 carbon atoms; and
Y⁻ is an anion such as bromide, chloride, acetate, borate, citrate, tartrate, bisulfate, bisulfite, sulfate or phosphate.

Mention may be made more particularly of the dimethyldiallylammonium salt (for example chloride) homopolymer sold, for example, under the name Merquat 100 by Nalco (and homologues thereof of low weight-average molar masses) and the copolymers of diallyldimethylammonium salts (for example chloride) and of acrylamide, notably sold under the names Merquat 550 and Merquat 7SPR;
(8) quaternary diammonium polymers comprising repeating units of formula:

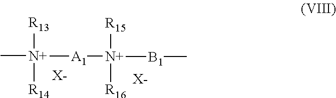

in which formula (VIII):
R₁₃, R₁₄, R₁₅ and R₁₆, which may be identical or different, represent aliphatic, alicyclic or arylaliphatic radicals comprising from 1 to 20 carbon atoms or lower (preferably C₁-C₆)hydroxyalkylaliphatic radicals, or alternatively R₁₃, R₁₄, R₁₅ and R₁₆, together or separately, constitute, with the nitrogen atoms to which they are attached, heterocycles optionally comprising a second heteroatom other than nitrogen, or alternatively R₁₃, R₁₄, R₁₅ and R₁₆ represent a linear or branched C₁-C₆ alkyl radical substituted with a nitrile, ester, acyl or amide group or a group —CO—O—R₁₇—D or —CO—NH—R₁₇—D where R₁₇ is an alkylene and D is a quaternary ammonium group;
A₁ and B₁ represent divalent polymethylene groups comprising from 2 to 20 carbon atoms which may be linear or branched, and saturated or unsaturated, and which may contain, linked to or inserted in the main chain, one or more aromatic rings, or one or more oxygen or sulfur atoms or sulfoxide, sulfone, disulfide, amino, alkylamino, hydroxyl, quaternary ammonium, ureido, amide or ester groups; and
X⁻ denotes an anion derived from a mineral or organic acid;
it being understood that A₁, R₁₃ and R₁₅ can form, with the two nitrogen atoms to which they are attached, a piperazine ring;
in addition, if A₁ denotes a linear or branched, saturated or unsaturated alkylene or hydroxyalkylene radical, B₁ can also denote a group (CH₂)ₙCO—D—OC—(CH₂)ₙ— in which D denotes:
a) a glycol residue of formula —O—Z—O—, in which Z denotes a linear or branched hydrocarbon-based radical or a group corresponding to one of the following formulae: —(CH₂—CH₂—O)ₓ—CH₂—CH₂— and —[CH₂CH(CH₃)—O]_{y}—CH₂—CH(CH₃)—, where x and y denote an integer from 1 to 4, representing a defined and unique degree of polymerization or any number from 1 to 4 representing an average degree of polymerization;
b) a bis-secondary diamine residue, such as a piperazine derivative;
c) a bis-primary diamine residue of formula: —NH—Y—NH—, where Y denotes a linear or branched hydrocarbon-based radical, or alternatively the divalent radical —CH₂—CH₂—S—S—CH₂—CH₂—; or
d) a ureylene group of formula: —NH—CO—NH—.

Preferably, X is an anion, such as chloride or bromide. These polymers have a number-average molar mass (Mn) generally of between 1000 and 100 000.

Mention may be made more particularly of polymers which are constituted of repeating units corresponding to the formula:

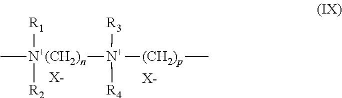

in which formula (IX) R₁, R₂, R₃ and R₄, which may be identical or different, denote an alkyl or hydroxyalkyl radical containing from 1 to 4 carbon atoms approximately, n and p are integers ranging from 2 to 20 approximately, and X is an anion derived from a mineral or organic acid.

A compound of formula (IX) that is particularly preferred is the one for which R₁, R₂, R₃ and R₄ represent a methyl radical and n=3, p=6 and X=Cl, which is known as Hexadimethrine chloride according to the INCI (CTFA) nomenclature;
(9) polyquaternary ammonium polymers comprising units of formula (X):

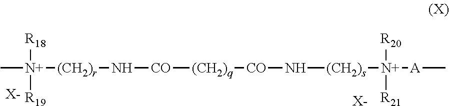

in which formula (X):
R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH radical, where p is equal to 0 or to an integer of between 1 and 6, with the proviso that R₁₈, R₁₉, R₂₀ and R₂₁ do not simultaneously represent a hydrogen atom,
r and s, which may be identical or different, are integers between 1 and 6,
q is equal to 0 or to an integer between 1 and 34,
X⁻ denotes an anion, such as a halide, and
A denotes a dihalide radical or preferably represents —CH₂—CH₂—O—CH₂—CH₂—.

Examples that may be mentioned include the products Mirapol® A 15, Mirapol® AD1, Mirapol® AZ1 and Mirapol® 175 sold by Miranol;
(10) quaternary polymers of vinylpyrrolidone and of vinylimidazole, for instance the products sold under the names Luviquat® FC 905, FC 550 and FC 370 by BASF;
(11) polyamines such as Polyquart® H sold by Cognis, which is referenced under the name Polyethylene Glycol (15) Tallow Polyamine in the CTFA dictionary;
(12) polymers including in their structure:
(a) one or more units corresponding to formula (A) below:

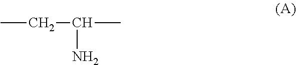

(b) optionally one or more units corresponding to formula (B) below:

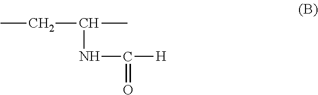

In other words, these polymers may be notably chosen from homopolymers or copolymers including one or more units derived from vinylamine and optionally one or more units derived from vinylformamide.

Preferably, these cationic polymers are chosen from polymers including, in their structure, from 5 mol % to 100 mol % of units corresponding to formula (A) and from 0 to 95 mol % of units corresponding to formula (B), preferentially from 10 mol % to 100 mol % of units corresponding to formula (A) and from 0 to 90 mol % of units corresponding to formula (B).

These polymers may be obtained, for example, by partial hydrolysis of polyvinylformamide. This hydrolysis may take place in acidic or basic medium.

The weight-average molecular mass of said polymer, measured by light scattering, may range preferably from 1000 to 3 000 000 g/mol, more preferentially from 10 000 to 1 000 000 and even more particularly from 100 000 to 500 000 g/mol.

The cationic charge density of these polymers may preferably range from 2 meq/g to 20 meq/g, more preferentially from 2.5 to 15 meq/g and more particularly from 3.5 to 10 meq/g.

The polymers including units of formula (A) and optionally units of formula (B) are notably sold under the name Lupamin by BASF, for instance, in a non-limiting manner, the products sold under the names Lupamin 9095, Lupamin 5095, Lupamin 1095, Lupamin 9030 (or Luviquat 9030) and Lupamin 9010.
(13) and mixtures thereof.

Even more preferentially, the cationic galactomannan gum(s) in the composition are chosen from cationic guar gums.

Preferably, the total content of cationic galactomannan gum(s) ranges from 0.01% to 10% by weight, more preferentially from 0.05% to 5% by weight, even more preferentially from 0.1% to 3% by weight, even better still from 0.1% to 1% by weight, relative to the total weight of the composition.

Preferably, the total content of cationic guar gum(s), when they are present in the composition, ranges from 0.01% to 10% by weight, more preferentially from 0.05% to 5% by weight, even more preferentially from 0.1% to 3% by weight, even better still from 0.1% to 1% by weight, relative to the total weight of the composition.

Preferably, the total content of additional cationic polymer(s), when they are present in the composition, ranges from 0.01% to 10% by weight, more preferentially from 0.05% to 5% by weight, even more preferentially from 0.1% to 3% by weight and better still from 0.1% to 1% by weight relative to the total weight of the composition.

### Particles

Preferably, the composition according to the present invention also comprises one or more particles.

The term “particles” means small fractionated objects formed from solid particles that are aggregated together, of variable shapes and sizes. They may be in regular or irregular form. They may in particular be in spherical form (such as granules, granulates or beads) or in square, rectangular or elongated form such as sticks. Spherical particles are most particularly preferred. The size of the particles may advantageously be, in the largest dimension thereof, between 1 nm and 5 mm, preferably between 10 nm and 2 mm, more preferentially between 100 nm and 100 μm and even better still between 1 μm and 50 μm. These particle sizes may be measured with a laser particle size analyzer (for example by means of the Brookhaven BI90).

Preferably, the particles according to the invention are chosen from opacifiers such as mica and glycol distearate, pigments such as titanium dioxide, fragments of one or more plants, and mixtures thereof.

For the purposes of the present invention, the term “fragment” means a piece or a part of a plant, obtained, for example, by tearing or milling the plant, or by cutting up said plant.

For the purposes of the present invention, the expression “fragments of one or more plants” is intended to mean that the composition comprises either several fragments of one and the same species of plant, or that the composition comprises several fragments of several different species of plants.

More particularly, the particles are visually in suspension stably and uniformly, in the medium, preferably cosmetic medium, of the composition according to the invention.

When the composition according to the invention comprises one or more particles, the suspending properties of the composition according to the invention make it possible to maintain the particles in suspension stably and uniformly.

The viscosity of the composition according to the invention may be evaluated by determining, at a temperature of 25° C., the flow time of an amount of product (for example 90 g of poured product) through a calibrated orifice using a Ford cup viscometer, equipped with a Ford cup with an orifice diameter of 8 mm (cF8).

Preferably, the flow time of the composition according to the invention is greater than or equal to 30 seconds, in particular greater than or equal to 50 seconds; in particular between 30 and 300 seconds, or even between 50 and 250 seconds, even better still between 50 and 120 seconds.

Preferably, the composition according to the invention also comprises at least one inorganic salt, and more preferentially sodium chloride.

For the purposes of the present application, the inorganic salt(s) are different from the anionic surfactants (i) and from the amphoteric or zwitterionic surfactants (ii), from the particles, and more particularly from all the ingredients described above.

Preferably, the total content of inorganic salt(s) present in the composition according to the invention ranges from 0.01% to 5% by weight, more preferentially from 0.05% to 3% by weight, and even more preferentially from 0.1% to 2% by weight relative to the total weight of the composition.

Preferably, the composition according to the invention comprises water, that is to say that the medium of the composition is aqueous or aqueous-alcoholic.

More preferentially, the total water content of the composition according to the invention is between 20% and 98% by weight, more preferentially between 50% and 96% by weight, even more preferentially between 60% and 95% by weight, even better still between 70% and 90% by weight, relative to the total weight of the composition.

The composition according to the invention may also comprise at least one organic solvent.

For the purposes of the invention, it is understood that the organic solvents are liquid at 25° C. and at atmospheric pressure.

By way of examples of organic solvent, use may particularly be made of those which are water-soluble, such as C₁-C₇ alcohols, and in particular C₁-C₇ aliphatic or aromatic mono alcohols, C₃-C₇ polyols and C₃-C₇ polyol ethers, which can be used alone or as a mixture with water. Advantageously, the organic solvent(s) may be chosen from ethanol, isopropanol, propylene glycol, hexylene glycol and glycerol, and mixtures thereof.

Preferably, the composition according to the invention comprises at least one C₂-C₇, more preferentially C₂-C₆, polyol, and even more preferentially propylene glycol.

Preferably, when the organic solvent(s) are present in the composition according to the invention, the total content of C₂-C₇ organic solvent(s) is between 0.01% and 10% by weight, more preferentially between 0.05% and 5% by weight, and even more preferentially between 0.1% and 1% by weight, relative to the total weight of the composition.

Preferably, when the composition according to the invention comprises water, the pH of the composition is between 3.0 and 9.0, more preferentially between 3.5 and 8.0, even more preferentially between 4.0 and 7.0, and even better still between 4.5 and 6.5.

The pH of the composition according to the invention may be adjusted to the desired value by means of basifying agents (such as monoethanolamine) and/or of acidifying agents (such as citric acid).

The composition according to the invention may optionally also contain one or more additives used in cosmetics, such as fragrances, thickeners or dyes.

These additives may be present in the composition according to the invention in an amount ranging from 0 to 20% by weight, relative to the total weight of the composition.

A person skilled in the art will take care to select these optional additives and amounts thereof so that they do not harm the properties of the compositions of the present invention.

The composition according to the invention may advantageously be in the form of a shampoo.

A subject of the present invention is also a process for treating, in particular cosmetically treating, keratin fibres, in particular human keratin fibres such as the hair, comprising at least one step of applying a composition as defined above to said keratin fibres.

Preferably, the process according to the invention is a process for washing and/or conditioning human keratin fibres, more particularly the hair, comprising at least one step of applying a composition as defined above to said human keratin fibres.

According to one preferred embodiment of the invention, a step of rinsing the keratin fibres is performed after the step(s) of applying a composition according to the invention to said fibres.

A step of drying the keratin fibres may be envisaged in the process according to the invention, especially using a heating means such as a hair dryer, a straightening iron, a steam iron or a heating hood; the heating means possibly heating to a temperature ranging from 35° C. to 230° C., preferably from 50° C. to 120° C.

A subject of the invention is also the use of the composition according to the invention as described above for treating keratin fibres, preferably for washing and/or conditioning keratin fibres.

The examples that follow serve to illustrate the invention without, however, being limiting in nature.

### EXAMPLES

### Example 1

Compositions A1, A2 and A3 according to the invention are prepared from the ingredients indicated in the table below, the amounts of which are expressed as weight percentages of active material (AM).

TABLE 1

| Ingredients | A1 | A2 | A3 |
|---|---|---|---|
| Sodium lauryl sulfate | 6.7 | 6.5 | 6.7 |
| Cocamidopropylbetaine | 2.7 | 2.8 | 1.7 |
| Cocobetaine | 0.1 | 0.1 | 0.1 |
| Capryloyl/caproyl methyl glucamide | 1.0 | 1.4 | 1.0 |
| Glyceryl oleate | 0.4 | 0.4 | 0.4 |
| Coco glucoside | 0.26 | 0.26 | 0.26 |
| Hydroxypropyl guar | 0.38 | 0.38 | 0.38 |
| hydroxypropyltrimonium chloride | | | |
| Sodium chloride | 0.5 | 0.5 | 1.2 |
| Glycol distearate | 0.5 | 0.5 | 0.5 |
| Carbomer | 0.1 | 0.1 | 0.1 |
| Preserving agents | qs | qs | qs |
| Propylene glycol | 0.1 | 0.2 | 0.1 |
| pH Agent | qs | qs | qs |
| | pH = 4.8 | pH = 4.8 | pH = 5.3 |
| Water | qs 100 | qs 100 | qs 100 |
| Weight ratio of the total content of | 2.4 | 2.2 | 3.7 |
| anionic surfactant(s) (i) to the total | | | |
| content of amphoteric or zwitterionic | | | |
| surfactant(s) (ii) | | | |

It was observed that the hair treated with one of compositions A1, A2 and A3 according to the invention is particularly clean, easy to disentangle, supple and soft to the touch.

In addition, compositions A1, A2 and A3 according to the invention generate a foam of good quality and in large amount.

### Example 2

Comparative composition B was prepared from the ingredients indicated in the table below, the amounts of which are expressed as weight percentages of active material (am).

TABLE 2

| | B |
|---|---|
| Ingredients | Composition |
| Sodium lauryl sulfate | 5.90 |
| Cocobetaine | 1.61 |
| Glyceryl oleate | 0.53 |
| Coco glucoside | 3.51 |
| Hydroxypropyl guar | 0.40 |
| hydroxypropyltrimonium chloride | |
| Sodium chloride | 1.35 |
| Glycol distearate | 0.50 |
| Preserving agents | qs |
| pH agent | qs pH = 5.3 |
| Water | qs 100 |
| Weight ratio of the total content of | 3.7 |
| anionic surfactant(s) (i) to the total | |
| content of amphoteric or zwitterionic | |
| surfactant(s) (ii) | |

### 2.1 Smoothness to the Touch:

The smooth nature of the hair treated with compositions A1 and A2 of Example 1 (invention) or composition B (comparative) was evaluated.

Compositions A1, A2 and B were respectively applied to 1 g locks of moderately sensitized Caucasian hair (AS20) 27 cm long, in a ratio of 0.4 gram of composition/gram of hair.

The smooth nature was then evaluated for each of the (dry or wet) treated hair locks, by measuring the friction effort (i.e. friction work in mJ) by means of a Lloyd Instruments tensile-compression testing machine (Ametek®).

More particularly, the friction effort between the lock to be evaluated (dry or wet) and two rollers of the tensile-compression testing machine which pass along the lock, from the root to the ends, a constant speed, and which simulate the fingers of the consumer, is measured in mJ.

The smoother the lock of hair is to the touch, the lower the friction effort measured.

The results are collated in the table below.

TABLE 3

| | Friction work on | Friction work on |
|---|---|---|
| | wet hair | dry hair |
| Compositions | (mJ) | (mJ) |
| A1 (invention) | 96.9 ± 9.3 | 134.2 ± 11.3 |
| A2 (invention) | 92.5 ± 10.4 | 131.99 ± 7.72 |
| B (comparative) | 134.3 ± 9.7 | 255.2 ± 18.6 |

It is observed that the hair treated with composition A1 or composition A2 according to the invention exhibits a better smoothness to the touch than the hair treated with comparative composition B.

### 2.2 Stability:

The stabilities of compositions A1 and A2 according to the invention and comparative composition B were evaluated.

For that, compositions A1, A2 and B, white in colour and with a homogeneous appearance, were placed in a vessel for 2 months at a temperature of 45° C. and in the dark.

After the two months of storage at 45° C., it was noted that compositions A1 and A2 according to the invention kept a homogeneous appearance and was white in colour, without any phase separation phenomenon.

After the two months of storage at 45° C., it was noted that comparative composition B at that time exhibited phase separation and was brown in colour.

It appears that compositions A1 and A2 according to the invention exhibit better stability over time than comparative composition B.

### Example 3

A sensory evaluation of the hair treated with compositions A1 and A2 (invention) of Example 1 was carried out.

Compositions A1 and A2 according to the invention were respectively applied to locks of moderately sensitized Caucasian hair (AS20) in a ratio of 0.4 gram of composition/gram of hair.

A sensory evaluation of the conditioning properties of each treated lock dry, then wet, was carried out by 3 experts. For that, each expert compares one of the locks treated (with A1 or with A2) with a lock of hair treated with a commercial shampoo comprising a silicone and in the same ratio of 0.4 gram of composition/gram of hair.

Each expert then grades the locks of hair treated according to the invention, according to the following scores:
+3: very much better than with the commercial shampoo,
+2: much better than with the commercial shampoo,
+1: better than with the commercial shampoo,
+0.5: slightly better than with the commercial shampoo,
0: similar to the lock treated with the commercial shampoo,
−0.5: slightly worse than with the commercial shampoo,
−1: worse than with the commercial shampoo,

The results are collated in the table below.

TABLE 4

| | Properties | A1 | A2 |
|---|---|---|---|
| Wet hair | Disentangling | +1 | +3 |
| | Smoothness | +1 | +1 |
| | Suppleness | +1.5 | +1 |
| | Coating | +1 | +2 |
| Dry hair | Smoothness | +1.5 | +1 |
| | Suppleness | +2 | +0.5 |

It is noted that the hair treated with composition A1 or A2 according to the invention exhibits conditioning properties that are at least equivalent to, and even better than, in terms of certain properties, the hair treated with the commercial shampoo based on silicone.

### Example 4

Compositions C1, C2 and C3 according to the invention, and also comparative composition C′, are prepared from the ingredients indicated in the table below, the amounts of which are expressed as weight percentages of active material (AM).

TABLE 5

| | C1 | C2 | C3 | C' |
|---|---|---|---|---|
| | inven- | inven- | inven- | inven- |
| Ingredients | tion | tion | tion | tion |
| Sodium lauryl sulfate | 6.6 | 5.4 | 6 | 7.8 |
| Cocamidopropylbetaine | 2.7 | 3.9 | 3.3 | 1.5 |
| Cocobetaine | 0.1 | 0.1 | 0.1 | 0.1 |
| Capryloyl/caproyl | 1.0 | 1.0 | 1.0 | 1.0 |
| methyl glucamide | | | | |
| Glyceryl oleate | 0.4 | 0.4 | 0.4 | 0.4 |
| Coco glucoside | 0.26 | 0.26 | 0.26 | 0.26 |
| Hydroxypropyl guar | 0.38 | 0.38 | 0.38 | 0.38 |
| hydroxypropyltrimonium | | | | |
| chloride | | | | |
| Sodium chloride | 0.5 | 0.5 | 0.5 | 0.5 |
| Glycol distearate | 0.5 | 0.5 | 0.5 | 0.5 |
| Carbomer | 0.1 | 0.1 | 0.1 | 0.1 |
| Preserving agents | qs | qs | qs | qs |
| Propylene glycol | 0.1 | 0.1 | 0.1 | 0.1 |
| pH Agent | qs | qs | qs | qs |
| | pH = 4.8 | pH = 4.8 | pH = 4.8 | pH = 4.8 |
| Water | qs 100 | qs 100 | qs 100 | qs 100 |
| Total content of anionic | 9.4 | 9.4 | 9.4 | 9.4 |
| surfactant(s) and of | | | | |
| amphoteric and | | | | |
| zwitterionic surfactant(s) | | | | |
| Weight ratio of the total | 2.4 | 1.3 | 1.8 | 4.9 |
| content of anionic | | | | |
| surfactant(s) (i) to the | | | | |
| total content of | | | | |
| amphoteric or zwitter- | | | | |
| ionic surfactant(s) (ii) | | | | |

### 4.1 Smoothness to the Touch and Disentangling:

The compositions above are applied in a standardized manner to pre-wetted, moderately sensitized (alkaline solubility=20%, AS 20) locks, while massaging the locks for 15 sec (6 passes between the fingers) in a proportion of 1 g/2.7 g of hair.

After a leave-on time of 1 minute, the hair is rinsed for 20 sec (25 passes between the fingers).

The hair is then run dry and evaluated while still wet.

The impact of the invention on the performance results in terms of smooth feel was evaluated on wet hair by 5 experts, in a blind test, on a score scale ranging from 0 (poor) to 8 (very very good). In order to evaluate the smooth feel, the expert takes the lock between the thumb and index finger and slides the fingers along the lock from the upper part to the ends.

The expert evaluates whether the hair is soft, whether or not it exhibits rough patches, whether or not it catches the fingers, and whether it feels uniform.

The results are collated in the table below.

TABLE 6

| Smooth | C1 | C2 | C3 | C′ |
|---|---|---|---|---|
| feel | invention | invention | invention | comparative |
| Expert 1 | 4 | 6 | 5 | 3 |
| Expert 2 | 4 | 5 | 5 | 2 |
| Expert 3 | 4 | 8 | 6 | 4 |
| Expert 4 | 4 | 8 | 6 | 4 |
| Expert 5 | 4 | 8 | 6 | 0 |
| Mean | 4 | 7 | 5.6 | 2.6 |
| Standard | 0 | 1.2 | 0.5 | 1.3 |
| deviation | | | | |

The experts judged that the smooth feel conferred by compositions C1, C2 and C3 according to the invention was improved compared to that of the comparative composition C′.

The impact of the invention on the performance results in terms of disentangling was evaluated on wet hair by 1 expert, in a blind test, on a score scale ranging from 0 (poor) to 8 (very good).

To evaluate the disentangling, the expert evaluates the ease with which a fine comb passes through the hair, while sliding it from the root to the end.

The results are collated in the table below.

TABLE 7

| C1 | C2 | C3 | C′ |
|---|---|---|---|
| invention | invention | invention | comparative |
| 7 | 7 | 7 | 0 |

The hair treated with compositions C1, C2 and C3 according to the invention is easier to disentangle compared with the hair treated with comparative composition C′.

### Example 5

Composition D1 according to the invention and comparative composition D2 are prepared from the ingredients indicated in the table below, the amounts of which are expressed as weight percentages of active material (am).

TABLE 8

| | D1 | D2 |
|---|---|---|
| Ingredients | (Inv) | (Comp) |
| Sodium lauryl sulfate | 6.7 | 6.7 |
| Cocamidopropylbetaine | 2.7 | 2.7 |
| Cocobetaine | 0.1 | 0.1 |
| Capryloyl/caproyl methyl glucamide | 0.37 | 0.37 |
| Glyceryl oleate | 0.4 | 0.4 |
| Coco glucoside | 0.26 | 0.26 |
| Hydroxypropyl guar | 0.38 | — |
| hydroxypropyltrimonium chloride | | |
| Polyquaternium-10 | — | 0.38 |
| Sodium chloride | 0.5 | 0.5 |
| Glycol distearate | 0.5 | 0.5 |
| Carbomer | 0.1 | 0.1 |
| Preserving agents | qs | qs |
| pH Agent | Qs pH = 4.8 | Qs pH = 4.8 |
| Water | qs 100 | qs 100 |
| Weight ratio of the total content of | 2.4 | 2.4 |
| anionic surfactant(s) (i) to the total | | |
| content of amphoteric or | | |
| zwitterionic surfactant(s) (ii) | | |

The compositions D1 and D2 above are applied in a standardized manner to pre-wetted, moderately sensitized (alkaline solubility=20%, AS 20) locks, while massaging the locks for 15 sec in a proportion of 1.5 g of composition per 1 g of hair.

The hair is then rinsed for 10 sec with water at 35° C. and then wrung out.

The wet hair is then evaluated (disentangling and suppleness) and dried in an oven before being evaluated again (smoothness to the touch).

### 5.1 Disentangling on Wet Hair:

A comb runs through the hair lock from root to tip. The earlier the comb gets stuck, the more difficult it will be to detangle the hair.

The distance between the root and where the comb got stuck is measured.

For hair treated with composition D1 according to the invention, the comb got stuck about 3 cm after the root (half of the lock).

For hair treated with comparative composition D2, the comb got stuck about 2 cm after the root (one third of the lock).

The hair treated with composition D1 according to the invention is easier to disentangle compared with the hair treated with comparative composition D2.

### 5.2 Suppleness on Wet Hair:

The impact of the invention on the performance results in terms of suppleness was evaluated on wet hair by 4 experts, in a blind test, on a score scale ranging from 0 (poor) to 8 (very very good).

In order to evaluate the suppleness, the expert takes the lock in his hands and tries to bend it. The expert assesses the hair's ability to bend easily, its malleability.

The results are collated in the table below.

TABLE 9

| | D1 | D2 |
|---|---|---|
| Suppleness | invention | comparative |
| Expert 1 | 4 | 2 |
| Expert 2 | 4 | 3 |
| Expert 3 | 4 | 3 |
| Expert 4 | 4 | 3 |
| Mean | 4 | 2.75 |
| Standard | 0 | 0.5 |
| deviation | | |

The hair treated with composition D1 according to the invention is more supple compared with the hair treated with comparative composition D2.

### 5.3 Smoothness to The Touch on Dry Hair

The impact of the invention on the performance results in terms of smooth feel was evaluated on dry hair by 4 experts, in a blind test, on a score scale ranging from 0 (poor) to 8 (very very good).

In order to evaluate the smooth feel, the expert takes the lock between the thumb and index finger and slides the fingers along the lock from the upper part (root) to the ends.

The expert evaluates whether the hair is soft, whether or not it exhibits rough patches, whether or not it catches the fingers, and whether it feels uniform.

The results are collated in the table below.

TABLE 10

| | D1 | D2 |
|---|---|---|
| Suppleness | invention | comparative |
| Expert 1 | 4 | 2 |
| Expert 2 | 4 | 2 |
| Expert 3 | 4 | 1 |
| Expert 4 | 4 | 3 |
| Mean | 4 | 2 |
| Standard | 0 | 0.8 |
| deviation | | |

The experts judged that the smooth feel conferred by composition D1 according to the invention was improved compared to that of the comparative composition D2.

1. Composition comprising:
(i) at least one anionic surfactant,
(ii) at least one amphoteric or zwitterionic surfactant,
(iii) at least one glucamide compound, and
(iv) at least one cationic galactomannan gum; and
in which the weight ratio of the total content of anionic surfactant(s) (i) to the total content of amphoteric or zwitterionic surfactant(s) (ii) is less than or equal to 4.3.

2. Composition according to claim 1, characterized in that the anionic surfactant(s) are chosen from sulfate anionic surfactants;
preferably from alkyl sulfates, alkyl ether sulfates, alkylamido ether sulfates, alkylaryl polyether sulfates, monoglyceride sulfates; the salts of these compounds, and mixtures thereof;
more preferentially from C₆-C₃₀, better still C₈-C₂₄, even better still C₁₀-C₂₄ or even C₁₂-C₂₂ alkyl sulfates, C₆-C₂₄, better still C₁₀-C₂₄, or even C₁₂-C₂₂ alkyl ether sulfates, preferably comprising from 1 to 20 ethylene oxide units, the salts of these compounds, and mixtures thereof;
even more preferentially from C₆-C₃₀, better still C₈-C₂₄, even better still C₁₀-C₂₄ or even C₁₂-C₂₂ alkyl sulfates.

3. Composition according to claim 1, characterized in that the amphoteric or zwitterionic surfactant(s) are chosen from (C₈-C₂₀)alkylbetaines, (C₈-C₂₀)alkylamido(C₃-C₈)alkylbetaines, (C₈-C₂₀)alkylamphoacetates, (C₈-C₂₀)alkylamphodiacetates and mixtures thereof; preferably from (C₈-C₂₀)alkylbetaines, (C₈-C₂₀)alkylamido(C₃-C₈)alkylbetaines and mixtures thereof.

4. Composition according to claim 1, characterized in that the weight ratio of the total content of anionic surfactant(s) (i) to the total content of amphoteric or zwitterionic surfactant(s) (ii) is between 1 and 4.3; preferably between 1.5 and 4.3; more preferentially less than or equal to 4; even more preferentially between 2 and 4; and even better still between 2.1 and 3.9.

5. Composition according to claim 1, characterized in that the total content of the anionic surfactant(s) and of amphoteric and zwitterionic surfactant(s) ranges from 4% to 40% by weight, preferably from 5% to 25% by weight, more preferentially from 6% to 20% by weight, even more preferentially from 7% to 15% by weight, and better still from 7.5% to 12% by weight relative to the total weight of the composition.

6. Composition according to claim 1, characterized in that the glucamide compound(s) are chosen from acylglucamides, preferably from acylglucamides having a hydrocarbon-based chain comprising from 4 to 30 carbon atoms, preferentially from 6 to 22, better still from 6 to 20 and even better still from 6 to 14 carbon atoms.

7. Composition according to claim 1, characterized in that the total content of glucamide compound(s) ranges from 0.01% to 10% by weight, preferably from 0.5% to 5% by weight and more preferentially from 0.8% to 3% by weight relative to the total weight of the composition.

8. Composition according to claim 1, characterized in that it also comprises at least one non-ionic surfactant; preferably at least one non-ionic surfactant chosen from monoesters of a fatty acid and of (poly)glycerol, alkyl(poly)glycosides, and mixtures thereof; more preferentially chosen from monoesters of a C₆-C₄₀ fatty acid and/or (poly)glycerol, (C₆-C₃₀)alkyl(poly)glycosides, and mixtures thereof.

9. Composition according to claim 1, characterized in that it also comprises:
(iv) at least one monoester of a fatty acid and of (poly)glycerol; preferably chosen from monoesters of a C₆-C₄₀ fatty acid and of (poly)glycerol; more preferentially from monoesters of a C₈-C₃₀ fatty acid and of (poly)glycerol; even more preferentially from monoesters of a C₈-C₃₀ fatty acid and of glycerol; and
(v) at least one alkyl(poly)glycoside; preferably chosen from (C₆-C₃₀)alkyl(poly)glycosides; more preferentially from (C₈-C₂₄)alkyl(poly)glycosides; even more preferentially from (C₈-C₁₈)alkyl(poly)glycosides; better still from coco glucosides, decyl glucosides, caprylyl/capryl glucosides, lauryl glucosides, and mixtures thereof.

10. Composition according to claim 1, characterized in that the cationic galactomannan gums are chosen from cationic guar gums.

11. Composition according to claim 1, characterized in that it is silicone-free.

12. Composition according to claim 1, characterized in that it further comprises one or more particles, preferably chosen from opacifiers, pigments, fragments of one or more plants, and mixtures thereof.

13. Composition according to claim 1, characterized in that it has a percentage of natural origin, within the meaning of international standard ISO 16128-2, which is greater than or equal to 90%; preferably greater than or equal to 92%; more preferentially greater than or equal to 94%.

14. Process for treating keratin fibres, preferably for washing and/or conditioning keratin fibres, comprising at least one step of applying a composition as defined in claim 1 to said keratin fibres.
